(19) United States
(12) Patent Application Publication
(10) Pub. No.: US 2015/0125406 A1
(43) Pub. Date: May 7, 2015

(54) TOPICAL SKIN CARE COMPOSITION

(71) Applicants: Marie C. Cassou Blaison, Fort Lauderdale, FL (US); Neisy Mirabal Padilla, Fort Lauderdale, FL (US)

(72) Inventors: Marie C. Cassou Blaison, Fort Lauderdale, FL (US); Neisy Mirabal Padilla, Fort Lauderdale, FL (US)

(73) Assignee: JAFER LIMITED, Road Town, Tortola (VG)

(21) Appl. No.: 14/397,824

(22) PCT Filed: Aug. 17, 2012
(86) PCT No.: PCT/US12/51417

§ 371 Date: Oct. 29, 2014

(51) Int. Cl. A61K 8/67; A61Q 17/04; A61K 8/55; A61Q 19/02; A61K 8/34

(52) U.S. Cl. CPC A61K 8/675; A61Q 19/02; A61K 8/347; A61K 8/55; A61Q 17/04
USPC 424/59; 424/62

(57) ABSTRACT

A topical skin care composition disclosed herein comprises a combination of complexion modifying components, and skin conditioning agents in a cosmetically acceptable vehicle and surprisingly helps achieve, restore and/or maintain a desirable enhanced uniform complexion color and moisturized tone on human skin. In a preferred embodiment, the skin care composition further includes skin protective agents to help ameliorate past sun-induced darkening of the skin and help protect the skin against further ravages from environmental ultraviolet radiation, such as sun-induced pigmentation and sun-induced aging.

### FIELD OF THE INVENTION

This invention relates to topical care of the skin, and in particular to compositions for achieving and maintaining a desirably uniform skin color and skin tone, and for ameliorating and protecting against the visible negative effects of cutaneous sun damage.

### BACKGROUND OF THE INVENTION

The appearance of pigmented freckles, blotches, and spots on the skin from exposure to sunlight and/or aging typically results in a complexion having a visibly uneven skin color or dull skin tone. Regardless of age, gender and ethnicity, the ravaging effect of sun and environmental conditions on the appearance and condition of the skin is generally undesirable. A fair, uniformly colored and brightly toned, moisturized complexion is generally desirable.

There is a strong desire, need, and demand for compositions that can be topically applied to the skin to reduce or minimize (i.e., lighten, whiten) undesirable dark pigmentation of the skin within a period of weeks, without resorting to skin-irritating chemicals or skin-abrasives.

The topical composition disclosed herein is cosmetically acceptable, physiologically tolerable by the skin, and surprisingly can help achieve, restore, and/or maintain a desirable uniform color and/or tone of the skin.

### SUMMARY OF THE INVENTION

In one aspect, the disclosed topical skin care composition helps achieve, restore and/or maintain a desirable uniform cutaneous color and moisturized tone on human skin. In another aspect, the disclosed topical skin care composition helps ameliorate past skin darkening effects of sunlight by lightening undesirably pigmented skin. In yet another aspect, the topical composition described herein helps protect the skin against further sun-induced pigmentation and sun-induced aging from environmental ultraviolet radiation.

The disclosed topical skin care composition comprises, consists essentially of, or consists of an aqueous cosmetically acceptable vehicle containing the following combination of complexion modifying components (a)-(c): (a) at least one 4-alkylresorcinol wherein the alkyl group has 1 to about 8 carbon atoms, preferably about 4 to about 6 carbon atoms; (b) at least one vitamin, selected from the group consisting of vitamin classes A, B; C, and E, preferably a vitamin B; and (c) is at least one unsubstituted or substituted alpha-aminoalkyl phosphinic acid. Component (c) preferably is a compound having the following structural Formula (I):

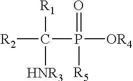

wherein R₁ is a hydrogen atom, a linear alkyl group, a branched alkyl group, or a thiazoline group; R₂ is a hydrogen atom, a linear alkyl group, or a branched alkyl group, wherein the linear or branched alkyl group optionally can include a substituent selected from a carboxyl, a hydroxyl, an amine, and a thiol group; R₃ is a hydrogen atom, a linear alkyl group, a branched alkyl group, an arylalkyl group, an acyl group, or an acyloxy group; R₄ is a hydrogen atom, a linear alkyl group, or a branched alkyl group; and R₅ is a hydrogen atom, a hydroxyl group, a linear alkyl group, or a branched alkyl group. In some preferred embodiments, component (c) is selected from the group consisting of 1-aminoethylphosphinic acid, 1-amino-3-methylbutylphosphinic acid, and a combination thereof.

Particularly preferred is a synergistic combination of components (a), (b) and (c), respectively comprising, consisting essentially of, or consisting of 4-hexylresorcinol, vitamin B₃ (niacinamide), and 1-amino-ethylphosphinic acid. In vitro testing of a composition comprising the foregoing combination of components (a), (b) and (c) exhibited surprising, statistically significant, synergistic inhibition of melanin production (whitening) as described in detail in Example 6 herein.

In some embodiments the cosmetic vehicle includes at least one skin conditioning agent selected from the group consisting of a humectant, and emollient, a moisturizer, and the like. The cosmetic vehicle of the skin care composition preferably includes at least one nonionic emulsifier to provide a composition in the form of an oil-in-water, lotion emulsion having a physiologically tolerable pH.

The skin care composition described herein provided, in a relatively short time period, a surprisingly enhanced complexion modifying, and skin conditioning effect.

### DETAILED DESCRIPTION OF THE INVENTION

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

The term “skin” includes the complexion of humans, and the term “complexion modifying component” refers to materials that help achieve and maintain a uniform pigmentation of the complexion and ameliorate uneven visual pigmentation of the complexion, by de-pigmenting, whitening and/or brightening the cutaneous color of the skin area to which the skin care composition is topically applied.

The disclosed topical skin care composition comprises, in an aqueous cosmetically acceptable vehicle, the following combination of complexion modifying components (a)-(c).

Component (a) comprises, consists essentially of, or consists of at least one 4-alkylresorcinol wherein the alkyl group has 1 to about 8 carbon atoms, preferably about 4 to about 6 carbon atoms, and most preferably 4-hexylresorcinol.

Component (b) comprises, consists essentially of, or consists of at least one vitamin, selected from the group consisting of vitamin classes A, B; C, and E. In some embodiments, component (b) comprises, consists essentially of, or consists of a B vitamin, preferably vitamin B₃ (niacinamide).

Component (c) comprises, consists essentially of, or consists of at least one unsubstituted or substituted alpha-aminoalkyl phosphinic acid. Component (c) preferably is a compound having the following structural Formula (I):

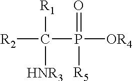

wherein R₁ is a hydrogen atom, a linear alkyl group, a branched alkyl group, or a thiazoline group; R₂ is a hydrogen atom, a linear alkyl group, or a branched alkyl group, wherein the linear or branched alkyl group optionally can include a substituent selected from a carboxyl, a hydroxyl, an amine, and a thiol group; R₃ is a hydrogen atom, a linear alkyl group, a branched alkyl group, an arylalkyl group, an acyl group, or an acyloxy group; R₄ is a hydrogen atom, a linear alkyl group, or a branched alkyl group; and R₅ is a hydrogen atom, a hydroxyl group, a linear alkyl group, or a branched alkyl group. In a preferred compound of Formula (I), R₁ is a hydrogen atom, a (C₁-C₄)alkyl group or a thiazoline group; each of R₂ and R₃ is a hydrogen atom; and R₅ is a hydrogen atom or a hydroxyl group. In a more preferred compound of Formula (I), R₁ is a (C₁-C₄) alkyl group, and each of R₂, R₃, R₄ and R₅ is a hydrogen atom.

In some embodiments, component (c) is selected from the group consisting of 1-aminoethylphosphinic acid, 1-amino-3-methylbutylphosphinic acid, and a combination thereof.

The amount (on an active weight basis) of component (a), calculated on the basis of the total weight of the composition, preferably is about 0.05 to about 3 parts by weight, more preferably about 0.1 to about 1.5 parts by weight, most preferably about 0.5 parts by weight. The amount of component (b) preferably is about 0.1 to about 5 parts by weight, more preferably about 0.5 to about 3 parts by weight, most preferably about 1 part by weight. The amount of component (c) preferably is about 0.05 to about 1 part by weight, more preferably about 0.1 to about 0.5 parts by weight, most about 0.2 parts by weight. As used herein, the term “parts by weight” as applied to any individual component is on the basis of the total composition of 100 parts by weight. Therefore, the term “parts by weight” is interchangeable with “percent by weight.”

In a particularly preferred embodiment, it was found that a combination (active weight basis) of about 0.5 parts by weight of 4-hexylresorcinol as component (a), about 1 part by weight niacinamide as component (b), and about 0.225 parts by weight of 1-aminoethylphosphinic as component (c), in the total composition surprisingly provided a synergistic inhibitory effect on melanin production compared against that of the individual components. A preferred cosmetic vehicle containing the foregoing combination of components in the foregoing ratio (a/b/c of about 1/2/0.5) surprisingly helped achieve, restore and/or maintain a desirable uniform skin color and moisturized tone on human skin within a period of about two to 4 weeks in an in vivo daily usage test.

The terms “cosmetically acceptable vehicle”, “cosmetic vehicle”, and grammatical variations thereof, are used interchangeably herein and include ingredients and cosmetic adjuvants suitable for compositions that are physiologically tolerable by the human skin when topically applied thereto. The term “cosmetic adjuvant” includes cosmetically useful product-stabilizing and product-finishing ingredients, well known and conventionally used in the cosmetic arts to maintain the physical stability of a composition, and the visible aesthetic appearance of a composition during storage and during the useful life of the composition. Cosmetic adjuvants that maintain the stability of products typically include a preservative, a metal-ion chelating agent, a perfume solubilizer, a pH modifier, a viscosity modifier, and/or ingredients that enhance the aesthetics and visual consumer appeal of the product (e.g., a fragrance, a product colorant, and the like).

Cosmetic ingredients, additives, products or materials, and cosmetic adjuvants, which can be employed in the skin care composition discussed herein are referred to by their commonly used chemical names or by the international nomenclature, (recognized by the European Union), commonly referred to as INCI name given them in any edition of the International Cosmetic Ingredient Dictionary and Handbook, (hereafter INCI Dictionary), or in any edition of the International Buyers' Guide, all published by the Personal Care Products Council (PCPC), Washington D.C. Numerous commercial suppliers of materials listed by INCI name, trade name, or both, can be found in any edition of the INCI Dictionary and in numerous commercial trade publications.

The term “skin conditioning agent” and grammatical variations thereof relate to lubricious and water-retaining (occlusive) materials that are cosmetically and physiologically tolerable, and include materials such as humectants, emollients, moisturizers, and the like, which are well known to those skilled in the cosmetic arts. Non-limiting examples of humectants include polyhydric alcohols (e.g., glycerin, propylene glycol, butylene glycol, hexylene glycol, and sugar alcohols, such as sorbitol, mannitol, galactitol, and the like). Examples of emollients include, without limitation thereto, esters of C₆-C₂₂ fatty acids (e.g., tri-esters of glycerin and a mixture of normal and branched chain C₁₀-C₁₈ fatty acids having the INCI name: C10-18 TRIGLYCERIDES), fatty alcohols, alkoxylated fatty alcohols, silicone fluids (volatile and nonvolatile), silicone copolyols, unsubstituted and substituted methyl polysiloxanes (e.g., dimethicone, cetyl dimethicone, bis-hydroxyethoxypropyl dimethicone), liquid hydrocarbons, (e.g., mineral oil), and the like. Non-limiting examples of moisturizers include organic polyols, salts of hyaluronic acid, salts of lactic acid, salts of pyrrolidone carboxylic acid, and water soluble polymers, such as polyethylene glycol. Those skilled in the art recognize that the properties of cosmetically acceptable skin conditioning ingredients may also contribute to more than one feature of the composition, such as viscosity or emulsion formation, for example.

The actual effective amount of individual or total skin conditioning agents present can be readily determined by the skilled formulator based on the residual desired tactile effect on the skin, without adversely affecting the physical stability of the emulsion composition, or its physiological acceptability. The total amount of skin conditioning agents are preferably in the range of about 1 to about 20 parts by weight, more preferably in the range of about 2 to about 15 parts by weight, based on the total weight of the skin care composition, without limitation thereto.

The compositions of this invention preferably are prepared as emulsions using anionic, amphiphilic, and nonionic emulsifying materials, which are well known in the art. Preferred emulsifiers are nonionic oil-in-water (o/w) emulsifiers selected from C₆-C₂₂ fatty acid esters of polyoxyethylene, monoglycerides, diglycerides, sorbitan esters, ethoxylated sorbitan esters, C₈-C₂₂ fatty alcohols, alkoxylated fatty alcohols, and combinations of two or more thereof. The term “alkoxylated” includes materials having ethyleneoxy and/or propyleneoxy oxide side chains with one or more such alkyleneoxy group in each side chain thereof. Particularly preferred nonionic emulsifiers are mixtures of C₁₆-C₁₈ fatty alcohols (INCI name: CETEARYL ALCOHOL), and an ethoxylated (20) glycerol ester of behenic acid (INCI name: TRIBEHENIN PEG-20 ESTERS), which are commercially available from suppliers found in the trade literature. The desirable amount of emulsifier can be readily determined by those skilled in the art, and is preferably in the range of about 1 to about 20 parts by weight of the final composition, but is not limited thereto.

Ultraviolet radiation absorbing materials, commonly referred to as UV filters or UV absorbers, are preferably included in the skin care composition as skin protective agents against continuing future undesirable uneven pigmentation of the skin from ongoing exposure to sunlight. UV filters preferably are UVA, UVB or broad spectrum absorbers, and are well known in the art. Example UV absorbers that are preferably included in the skin care composition are selected from the group consisting of HOMOSALATE (INCI name for 3,3,5-trimethylcyclohexyl 2-hydroxybenzoate); OCTISALATE (INCI name for ethylhexylsalicylate); OXYBENZONE (INCI name for Benzophenone-3); OCTOCRYLENE (INCI name for 2-ethylhexyl-2-cyano-3,3-diphenyl acrylate); AVOBENZONE (INCI name for butyl methoxydibenzoylmethane); and combinations thereof, without being limited thereto. Preferably, an effective amount of UV absorbing material present in the composition provides a calculated Sun Protective Factor (SPF) value of at least about 15, more preferably at least about 30. The actual amount of UV absorber present can be readily determined by those skilled in the art. The total amount of UV absorber, when present, preferably varies from about 1 to about 30 parts by weight, more preferably from about 2 to about 25 parts by weight, based on the weight of the composition.

Cosmetic adjuvant ingredients that may be present in the skin care compositions include synthetic or natural metal ion chelating agents, preservatives against microbiological deterioration, product colorants, and fragrances. Non-limiting examples of chelating agents include ethylenediaminetetraacetic acid (EDTA), citric acid, gluconic acid, cyclodextrins, phytic acid, carboxylic acids derived from monosaccharides, and salts thereof, such as sodium gluconate, glucaric acid, and the like. Non-limiting examples of product colorants, include opacifying and pearlizing materials (e.g., titanium-coated mica, titanium dioxide, and the like). The amount of cosmetic adjuvant ingredient need only be present in a quantity sufficient (q.s.) to effectively accomplish its respective function. Those skilled in the cosmetic arts recognize that cosmetic adjuvants may provide more than one benefits or function.

The aqueous skin care compositions preferably are formulated in the form of an oil-in-water emulsion, having a pourable, liquid viscosity, but are not limited thereto. Particularly preferred is a lotion emulsion composition having a physiologically tolerable pH in the range of about pH 4 to about pH 8, more preferably about pH 5 to about pH 6.5, most preferably about pH 5.5 to about pH 6, and having a viscosity in the range of about 4,000 to about 30,000 milli-Pascal seconds (mPa·s) measured at a temperature of about 25° C. with an LVF viscometer. Preferably, a skin care emulsion composition having a measured viscosity of about 10,000 mPa·s remains physically stable (i.e., no oil separation or discoloration) for at least six weeks storage at an ambient room temperature of about 25° C.

A generic example of a topical skin care composition is illustrated in Table 1 below:

TABLE 1

| | Parts by weight of |
|---|---|
| Ingredient(s) | total composition |
| Complexion modifying components: | 0.2-10 |
| (a), (b), and (c) | |
| Skin conditioning agents | 1-20 |
| (humectants, emollients, moisturizers) | |
| Skin protective agents (UV absorbers) | 0-30 |
| Emulsifier | 1-20 |
| Cosmetic adjuvants (viscosity modifying | q.s. |
| agents, preservative, fragrance, metal-ion | |
| chelating agents; pH adjusting agents) | |
| Water, deionized to 100 parts by weight | q.s. |

q.s. = quantity sufficient

The following examples are provided to illustrate preferred embodiments of the present invention, and are not meant to limit the scope of the invention.

### Example 1

Generic (A) and specific (B) examples of preferred embodiments of skin care compositions are illustrated in Table 2, where component (a) is 4-hexylresorcinol; component (b) is vitamin B₃ (niacinamide); and component (c) is 1-amino-ethylphosphinic acid. On an active weight basis, the ratio of components (a)/(b)/(c) in specific embodiment (B) is 1/2/0.5.

TABLE 2

| | |
|---|---|
| INGREDIENTS | PARTS BY WEIGHT |

| (INCI/Common Name) | A | B |
|---|---|---|
| 4-Hexylresorcinol (min. 99%) | 0.05-0.75 | 0.5 |
| Vitamin B₃ (Niacinamide) | 0.5-1.5 | 1 |
| 1-Amino-ethylphosphinic acid | 0.1-1.5 | 1 |
| (22.5%) Note (a) | | |
| Polyhydric alcohol | 0.01-5 | 4 Note (b) |
| Tribehenin PEG-20 Esters | 2-6 | 4.5 |
| Cetearyl alcohol NF | 1-5 | 2 |
| C10-18 Triglycerides | 1-5 | 2 |
| Cetyl dimethicone | 0-1 | 0.5 |
| Bis-Hydroxyethoxypropyl | 0-2 | 1.5 |
| dimethicone | | |
| Homosalate | 0-15 | 10 |
| Avobenzone | 0-5 | 2 |
| Octisalate | 0-8 | 5 |
| Octocrylene | 0-5 | 3 |
| Oxybenzone | 0-5 | 3 |
| Silica | 0-2 | 1.5 |
| Polyacrylamide (and) C13-14 | 0-2 | 0.5 |
| Isoparaffin (and) | | |
| Laureth-7 (and) water | | |
| Sodium hyaluronate | 0-0.5 | 0.01 |
| Microcrystalline cellulose and | 0-1 | 0.5 |
| cellulose gum | | |
| Xanthan gum | 0.1-0.5 | 0.3 |
| Preservative, metal ion chelating | q.s. | q.s. |
| agent, and/or fragrance | | |
| Sodium hydroxide and/or Citric | q.s. | q.s. |
| Acid to pH 5.5-6 | | |
| Water, deionized, to | q.s. | q.s. |
| 100 parts by weight | | |

Notes to Table 2:
(a) Commercially sold under the INCI name: AMINOETHYLPHOSPHINIC ACID (and) BUTYLENE GLYCOL (and) WATER as a 22.5% active concentration in aqueous solution, reportedly also containing 7.7% butylene glycol, 0.15% sodium salt of para-hydroxybenzoic acid, and 0.2% citric acid monohydrate.
(b) Equal parts by weight of butylene glycol and glycerin.

Composition B was a homogeneous, opaque white, pourable, oil-in-water emulsion.

### Example 2

### Skin Moisturization Measurement by Corneometry

This example illustrates the long-lasting skin moisturizing efficacy of composition of Example 1B as instrumentally measured by corneometic technique in a controlled in vivo study described below.

Method Criteria and Protocol.

Twenty clinically healthy female subjects ranging in age between 23 and 61 years old were included in the study. Pregnant females, and females having skin diseases, were excluded. Each one of the subjects was instructed not to use any topical preparations on the test areas starting from seven days prior to testing and until the end of the test. For cleansing during the run-in phase, only the use of water or a mild syndet (such as Eubos® flüssig-blau; Dr. Hobein, D-53340 Meckenheim-Merl, Germany) supplied by the testing facility was allowed.

Before application of the composition, the hydration state of the stratum corneum was measured at clearly defined sites of the inner forearm using a Corneometer MPA 5 CPU (S/N 09/372,310; probe SN 09/341,841, Courage and Khazaka, Cologne, Germany). The Corneometer registers the electrical capacitance of the skin surface, and is expressed digitally in arbitrary units (a.u.). The probe head (7×7 mm) consisting of a condenser was applied to the skin surface at constant pressure (3.5 N). The measuring principle is based on distinctly different dielectric constants of water (approximately 81) and most other materials (less than seven). For each subject, one randomly selected skin site was predefined as the test site and one site remained untreated as a control. Five measurements were performed on each predefined skin site and the mean was used to define the hydration state of the stratum corneum of the test or control skin area.

Controlled application of approximately 2 mg/cm² composition to the predefined skin site was made by a trained technician, followed by 30 seconds of massaging (gloved fingers) the composition into the skin. The subjects were instructed not to cover the test areas by clothing during the first 20 minutes after product application, and if the product was not absorbed into the skin completely, remaining liquid was removed with a soft paper towel by the facility staff member (not required in majority of subjects). Corneometer measurements were performed 2, 4, 6, 8 and 24 hours following the product application (adaptation time: 30 min., room temperature: 21±1° C., relative humidity: 50±5%). Subjects were asked to avoid bodily exertion and water contact during these periods.

Changes in the hydration values of the treated skin area were compared to changes in the hydration values of the untreated skin (control) area. Measurement data was automatically computerized and after validity check and quality assurance stored centrally in a database. Data evaluations were conducted using the software NAG® Statistical Add-Ins for Excel (NAG Ltd., United Kingdom), analyzed by Wilcoxon Rank test, and the 0.05 level was selected as the point of minimal acceptance of statistical significance. An increase in the measurement value corresponds to an increase in skin hydration.

Results.

Two hours after the application of the composition of Example 1-B, a steep, a statistically significant, (p<0.05), increase in skin hydration was observed in the skin area onto which the composition was applied as compared to changes in the untreated control area. Over time, the hydrating effect diminished, but the increased hydration level remained statistically significant in the Ex. 1-B treated skin area even at the final 24-hours measurement after the single application.

The mean corneometer readings compared to the base line control, for all participants is summarized in Table 3 and The increase in skin hydration by the composition of Example 1-B relative to the initial values and to the untreated control expressed as a % increase in skin hydration is summarized in Table 4.

TABLE 3
Mean Corneometer Reading (a.u.)

| Site | 2 hrs | 4 hrs | 6 hrs | 8 hrs | 24 hrs |
|---|---|---|---|---|---|
| Control | 0.4 | 0.6 | 0.4 | 0.4 | 0.4 |
| Ex-1B | 18.7 | 13.4 | 11.1 | 9.0 | 4.3 |

TABLE 4
% Increase in Skin Hydration (Moisturization)

| Comp. | 2 hrs | 4 hrs | 6 hrs | 8 hrs | 24 hrs |
|---|---|---|---|---|---|
| Ex-1B | 57.4 | 40.7 | 34.3 | 27.3 | 12.4 |

The data show that the composition of Example 1-B statistically significantly increased skin hydration after a single application and that a long-lasting (24-hour) moisturization effect was maintained. Based on the mean corneometer readings, the statistically significant positive hydrating effect (i.e., moisturizing efficacy) of the composition of Example 1-B was detected after 8 hours in 100% of the study participants, and after 24 hours, in 95% of the study participants.

### Example 3

### Complexion Color Modification Efficacy

This example illustrates the efficacy of a composition of Example 1-B in modifying the complexion to a desirable visual, uniform cutaneous color appearance within a one to two month in vivo usage period. Efficacy was instrumentally determined and subjectively evaluated as described below.

Method Criteria and Protocol.

Healthy female subjects having an untanned Fitzpatrick skin phototype of III or IV, who were at least 35 years old and had visible facial pigmentary blotches with a diameter greater than 3 millimeters (mm) were included in the study. Thirty subjects were selected for instrumental cutaneous color measurements to determine lightening and anti-blotch effects and 52 subjects were selected for subjective evaluation of the visual effects after a once-daily use of a composition of Example 1-B over the period of 28 to 56 days.

Excluded from the study were females who were pregnant or nursing; had changed, started or stopped oral contraceptive or any hormonal treatment less than 1.5 months of the study; had cutaneous pathology on the studied zones (eczema, etc.); used topical or systemic treatment during the previous weeks liable to interfere with the assessment of the efficacy of the studied composition; used depigmenting product less one month before the study; used, on the studied skin area, any facial mask, exfoliant product, or the like; had excessive exposure to sunlight or UV-rays within the previous month (or during the study); or who enrolled in any another clinical trial during the study period.

Restrictions During the Study.

The participant was permitted to use her usual cleansing and cosmetic products (after the composition of Example 1-B was applied on the face). The participant was instructed not to use scrub-type, or exfoliant-type products, or depigmenting products during the study and avoid intentional exposure to sunlight during the study or to apply a solar protection product. If the protocol was not respected and if the deviation was minor, the technician or the investigator warned the subject of the importance of respecting the prescribed protocol. If the subject persisted or if the deviation was major, the subject was declared non-complaint. In this case, the subject was removed from the study for non-compliance. Under normal conditions of use (facial application of the product at home), no compliance control could be carried out during the study. However, the subjects filled in every day a daily log and notice the number of use.

### Operational Aspect:

Day 0.

The subject came to the testing facility without having applied any products to the face since the previous evening. She read, signed and dated the information sheet (instructions on the product use and restrictions related to the study) and informed consent forms. Two zones were defined on the face: one zone with spots and one “normal” zone without spots. The skin color was instrumentally measured using a MINOLTA CR321 Chromameter® on each of the two zones defined. The subject was instructed to apply the composition once-daily (in the morning) to the face, under normal conditions of use, during a period of 56 days and given a daily log form to fill out.

Days 28 and 56.

The subject returned to the testing facility without having applied any products to the face since the previous evening, the last application of composition of Example 1-B having been applied the morning before. New chromameter measurements of the skin color were made on the two zones defined on Day 0. The subject filled in a subjective evaluation questionnaires on Day 28 and 56.

Skin colorimetric measurement was made using a MINOLTA CR321 Chromameter®, equipped with a 3 mm diameter head. The Chromameter® converts colors perceived by man to a digital code composed of a luminance parameter, L* for clarity (from dark to light); and two chrominance parameters: a* for the green-to-red spectrum, and b*: for the blue-to-yellow spectrum. It is, therefore, possible to express in the slightest details the differences between two cutaneous zones that appear to be the same color. After a calibration phase, measurements were done directly on the skin using a pulsed Xenon light source and a dual beam system designed to measure the light transmitted and to correct any slight deviation.

The parameters for L* (luminance) and b* (cutaneous melanin yellow color) were used to evaluate pigmentary skin blotch by calculating the subject's Individual Typological Angle)(ITA°, which defines the skin pigmentation degree of a subject as measured chromametrically. The ITA° was calculated from the skin clarity (L*) and the melanin parameter (b*) according to the following formula: ITA°=[Arc tan ((L*−50)/b*)]×180/π. The higher the ITA° value, the lighter the skin complexion color.

### Data Analysis:

The raw variations (Δ) and in percentage on the means (Δ %) were calculated according to the following formulas:

$$Δ = ( {TZ}_{ti} - {TZ}_{t 0} )$$

$$Δ \% = \frac{( {TZ}_{ti} - {TZ}_{t0} )}{{TZ}_{t 0}} \times 100$$

where TZ is the value obtained on the treated zone(s), t0 is the value before application, and ti is the value at each measurement time after application. The percentage of the variation (Δ %) is expressed in percentage of the variation on the measurements zone (TZₜᵢ−TZₜ₀). These variations were balanced at the initial value TZₜ₀ (before application). The expression (Δ %), therefore, gives the variation, in percentage, on the measurement's zone compared to the initial conditions (TZₜ₀).

The data were statistically analyzed to determine the significance of the measurement variations obtained under the effect of the tested product. The comparison was on the values obtained before and at the different times of kinetics after treatment. Data were analyzed, using EXCEL version 2003 software, with a paired t-test. This method tested whether the mean of sample differences between pairs of data was significantly different from the hypothetical mean, zero under the null hypothesis (H0). The alternative hypothesis (h1) was that the average difference was either greater or less than 0 (two-tailed test). Before carrying out a test, a type 1 error of 5% was chosen (which corresponds to the risk of rejecting a true null hypothesis). If p≦0.05, H0 was rejected (there was a significant difference between before and after the treatment). If p≦0.05, H0 was accepted, (the mean was not different from 0 so data did not show a significant difference between before and after the treatment).

An increase in the L* parameter value characterized a lightening of the color of the skin (from dark to light). A decrease in the cutaneous melanin parameter b* (from the blue to yellow) characterized a decrease in the yellow constituent of the skin. An increase in the ITA° value characterized a decrease in the skin pigmentation.

### Results.

Skin Lightening Efficacy:

In comparison with the initial state, the composition of Example 1-B provided a significant lightening effect of the facial skin as summarized below.

The L* parameter increased significantly by about +1% on average, after 28 and 56 days of once daily use of the composition:(respectively by +0.49±0.09 (p<0.001) and +0.83±0.14 (p<0.001)). This effect was observed on 83% of the subjects.

The ITA° parameter increased significantly of by about +9% and +13% on average, after 28 and 56 days of once daily use of the composition (respectively by about +2±0 (p<0.001) and +3±1 (p<0.001)). This effect was observed in 77% and 67% of the subjects.

Anti-Blotch Efficacy:

In comparison with the initial state, the composition of Example 1-B provided a significant anti-blotch effect of the facial skin as summarized below. The L* parameter increased significantly by about +1% and +2% on average, after 28 and 56 days of once daily use of product, respectively by +0.61±0.08 (p<0.001) and +1.09±0.11 (p<0.001). This effect was observed in 90% and 97% of the subjects.

The b* parameter decreased significantly by about −3% on average after 56 days of once daily use of product (−0.39±0.14 (p=0.010)). This effect was observed on 67% of the subjects;

The ITA° parameter increased significantly of +48% and +86% on average, after 28 and 56 days of once daily use of product (respectively +2±0 (p<0.001) and +4±0 (p<0.001)). This effect was observed in 73% and 93% of the subjects.

Subjective Evaluations:

The answers given by 52 subjects to a subjective evaluation questionnaire were used to evaluate the organoleptic characteristics, efficacy and tolerance of the composition of this invention. A summary of the overall comments is shown in Tables 5 and 6.

TABLE 5

| Characteristic | Comment | No. Subjects |
|---|---|---|
| Global appreciation | Very Pleasant/Pleasant | 99% |
| Aspect | Very Pleasant/Pleasant | 89% |
| Texture | Very Pleasant/Pleasant | 98% |
| Non-Greasy | Agree/Rather Agree | 91% |
| Product Absorbs by Skin | Very Quickly/Quickly | 98% |

TABLE 6

| PRODUCT EFFICACY | | Day 28/56 |
|---|---|---|
| Skin Softer | More to Much More | 75%/80% |
| Skin Smoother | More to Much More | 81%/77% |
| Skin Supple | More to Much More | 77%/79% |
| Skin Moisturized | More to Much More | 75%/87% |
| Skin Nourished | More to Much More | 82%/89% |
| Skin Luminous | More to Much More | 62%/77% |
| Skin Younger Looking | More to Much More | 46%/67% |
| Complexion More Even | | 98%/96% |
| Skin Tone Lighter | | 96%/100% |
| Reduced Dark Spots | | 94%/96% |
| Reduced Blotch Color | Much clearer (17%) | 94%/92% |
| (clarity) | clearer (44%/54%) | |
| | slightly clearer | |
| | (33%/21%) | |
| Blotch Size | Much less (13%/17%); | 92%/96% |
| (large blotches) | less (35%/56%); | |
| | slightly less | |
| | (44%/23%) | |

No unpleasant or uncomfortable sensations were reported by any of the subjects during the use of the composition. After the 28 day period, 98% of the subjects reportedly perceived a general improvement in her skin ranging from light (29%) to moderate (46%) to substantial (23%); after the 56 day period, all (100%) of the subjects reportedly perceived a general improvement in her skin ranging from light (12%) to moderate (44%) to substantial (44%).

In comparison with the initial state of the skin, the composition of Example 1-B was judged to be efficacious as a skin care composition. As summarized below, the composition of this invention:

(1) induced a significant lightening effect: significant increase in L* parameter (+1% on average, effect observed in 83% of the subjects) and ITA° parameter (respectively of +9% and +13% on average effect observed in 77% and 67% of the subjects) after 28 and 56 days of once daily use;
(2) induced a significant anti-blotch effect: significant increase in L* parameter (+1% and +2% on average, effect observed in 90% and 97% of the subjects) and ITA° parameter (respectively of +48% and +86% on average effect, observed in 73% and 93% of the subjects) after 28 and 56 days of once daily use; and a significant decrease in b* parameter (−3% on average, effect observed in 67% of the subjects) after 56 days of once daily use; and
(3) satisfied the majority of the subjects for its organoleptic characteristics (i.e. global appreciation, aspect, texture, non-greasy, quick penetration) and for the skin care efficacy after 28 and 56 days of use (the skin was reportedly softer, smoother, more supple, more moisturized, more nourished, more luminous, younger looking, had a more even complexion tone, lighter skin tone, reduction of the number of dark spots and clearer, less number of large blotches).

### Example 4

### Skin Protective Efficacy

This example illustrates the in vivo skin protective efficacy of a composition of Example 1-B, using the International Sun Protection Factor (SPF) Test Method, (Colipa Guidelines, May 2006) to evaluate its static SPF value. For comparison, the composition was compared against the static SPF value of a P2 standard sunscreen product as set forth in the Colipa Guidelines.

Ten subjects, (6 male and 4 female subjects), were selected. Eight subjects had skin phototypes of Fitzpatrick Type I (always burns easily; never tans, sensitive) and two subjects had skin phototypes of Fitzpatrick Type II (always burns easily; tans minimally, sensitive). The subjects selected were at least 18 years of age (ranged from 19-60 years), had fair, uniformly-colored skin on the lower area of the back which would allow for discernable erythema; were free of any dermatological or systemic disorder which, in the opinion of the testing personnel, would interfere with the results of the study; were in good health, and had read, understood and signed a consent document in compliance with the described in 21 CFR 50.

Excluded from the study were individuals with dermatological problems or existing skin damage; taking medications with photosensitizing potential; pregnant and nursing females; with history of any form of cancer, or hepatitis or other blood disease; known sensitivity to cosmetics, skin care products or topical drugs; and individuals with recent sun exposure on the areas to be tested.

A Xenon Arc Solar Simulator lamp, which has a continuous light spectrum in the UVA and UVB range (290-400 nanometers), was utilized as the light source. The spectral output of the solar simulator was filtered to meet the spectral output requirements for testing Suscreen Drug Products for over-the-counter human use set out in the Proposed Amendment of Final Monograph, CFR Part 352.70(b) Light sources, (Federal Register, Vol. 72, No. 165, Aug. 27, 2007 and the Colipa Guidelines), the relevant disclosures of which are incorporated herein by reference.

### Test Protocol:

Day 1.

The individual reported to the testing facility. and a trained technician determined the initial Minimum Erythemal Dose (MEDu) on a predefined unprotected skin area as follows. A series of 5 UV radiation doses, expressed as Joules/square meter using the recommended geometric progression of 1.25, was administered to an unprotected location on the subject's back, between the belt-line and shoulder blades. The following 5 dose series, with X representing the amount of UV energy projected to produce the test subject's MEDu are shown in Table 7.

TABLE 7

| Dose 1 | Dose 2 | Dose 3 | Dose 4 | Dose 5 |
|---|---|---|---|---|
| 0.64X | 0.80X | 1.00X | 1.25X | 1.56X |

The subject was instructed to avoid additional UV exposure, and to avoid taking any photosensitizing medications until conclusion of the study.

Day 2.

The subject returned to the testing facility within 16 to 24 hours following the completion of the MEDu doses for evaluation of the response and to determine the subject's unprotected MED (MEDu) value. The MEDu value was the lowest level of UV dose that produced the first perceptible unambiguous erythema with defined borders using the following grading scale:

| | |
|---|---|
| − | No perceptible erythemal response |
| ? | Barely perceptible erythemal response |
| + | Unambiguous erythema with defined borders |
| ++ | Moderater erythema with sharp borders |
| +++ | Dark red erythema with sharp borders to |
| | evaluate the effects. |

Two test areas (10 cm×5 cm), as 50 square centimeter rectangles, were drawn in the designated location on the subject's predefined area of the back using a template and an indelible marker. The trained technician applied a composition of Example 1-B to one of the test areas and a P2 standard sunscreen in the adjacent test area. Each composition, in an amount of 2 mg/cm², was applied by “spotting” the product across the test area and gently spreading, using a finger cot, until a uniform film was applied to the entire test area. The applied product was allowed to dry a minimum of 15 minutes prior to UV exposure.

The technician administered a series of 5 UV radiation doses, expressed as Joules/square meter using the recommended geometric progression of 25% for the P2 Standard and 12% for the composition of Example 1-B, determined by the previously established MEDu from Day 1. For an expected SPF of 15 for the Static SPF P2 Standard, the doses applied were as previously shown in Table 7. For an expected SPF of 30 for the composition of Example 1-B, with X representing the expected SPF range of the test product, the doses applied were as shown in Table 8.

TABLE 8

| Dose 1 | Dose 2 | Dose 3 | Dose 4 | Dose 5 |
|---|---|---|---|---|
| 0.80X | 0.89X | 1.00X | 1.12X | 1.25X |

On Day 2, the technician also administered a second timed series of 5 UV doses, increasing in 25% increments to an unprotected area of the subject's back to determine the subject's second Day MEDu, the doses including the original MEDu from Day 1 as shown in Table 5.

Day 3.

The subject returned to the testing facility after 16 to 24 hours following completion of the UV doses from Day 2. The MED values for all sites that received UV doses, both protected and unprotected areas, were evaluated and recorded under a source of illumination that was a warm white light bulb that provided a level of illumination of at least 500 lux. The subject was seated when the test site was irradiated and when evaluated.

The study was conducted in a double-blinded manner. Neither the test subject nor the designated technician who evaluated the MED responses knew which formulation was applied to which site or what doses of UV radiation were administered, because different technicians performed the product application and administered the doses of UV radiation.

The SPF values were calculated for both the composition of Example 1-B and the P2 standard for each individual (I) subject by calculating the ratio of the MEDp (value produced in the sunscreen protected site) to the MEDu (value produced in the unprotected test area), using the calculation: MEDpi/MEDui=SPFi value. The SPF of the test product was the arithmetical mean of the individual SPFi values obtained from the total number (n) of subjects used, expressed to one decimal point: SPF=(SUM SPFi)/n. The “Label SPF” value of the tested sunscreen product was the mean SPF value rounded down to the next whole number.

### Results.

The composition of Example 1-B tested under Static sun protective conditions has a Static Mean SPF of 34.6 and a Static Label SPF of 34, whereas the P2 Standard had a Static Mean SPF of 16.9, (well within the allowable guidelines of 16.7±3.6. Thus the data from the ten subjects met the statistical criteria for validity. No adverse experiences were reported during this in vivo study.

### Example 5

### Skin Protective Efficacy

This example illustrates the in vivo skin protective efficacy of a composition of Example 1-B, using the procedures specified in the Japan Cosmetic Industry Association, JCIA-Measurement Standards for UVA Protection, 1999, using a xenon arc solar simulator as the UVA source. For comparison, the composition was compared against a JCIA UVA Certified Sunscreen Product.

Ten subjects, (2 males and 8 female subjects), were selected. Four subjects had skin phototypes of Fitzpatrick Type II (always burns easily; tans minimally) and six subjects had skin phototypes of Fitzpatrick Type III (burns moderately; tans gradually). The subjects selected were at least 18 years of age (ranged from 19-60 years), had fair, uniformly-colored skin on the lower area of the back which would allow for discernable pigment darkening response; were free of any dermatological or systemic disorder which, in the opinion of the testing personnel, would interfere with the results of the study; were in good health, and had read, understood and signed a consent document in compliance with the described in 21 CFR 50.

Excluded from the study were individuals with any visible skin disease at the study site which, in the opinion of the testing personnel, would interfere with the results of the study; taking medications, such as photosensitizers, antihistamines, analgesics or anti-inflammatory drugs; pregnant and nursing females; were over 60 years old, had a known sensitivity to cosmetics, skin care products or topical drugs; and individuals with recent sun exposure on the areas to be tested.

A Xenon Arc Solar Simulator lamp (Solar Light Co., Philadelphia Pa.), Model 601-300W, which provided a continuous emission spectrum in the UVA and UVB range (290-400 nanometers), was used as the light source. The ratio of UVA I (340 to 400 nanometers) to UVA II (320 to 340 nanometers) in the final beam was close to that of sunlight, i.e., emitted UVA II was 8 to 20 percent of the total UVA radiation. Optical radiation from 250 to 320 nanometers was less than 0.1 percent of the optical radiation between 320 and 400 nanometers. The spectral output of the solar simulator complied with the specifications for testing Suscreen Drug Products for over-the-counter human use set out in the Proposed Amendment of Final Monograph, CFR Part 352.70(b) Light sources, (Federal Register, Vol. 72, No. 165, Aug. 27, 2007) and met all JCIA requirements. UV radiation was monitored continuously during exposure using a PMA 2100 Dose control System equipped with a PMA 2113 UVA Detector (Solar Light Co.). The field of irradiation (test subsites) were 0.8 mm in diameter, and the solar simulator was measured by an appropriately calibrated spectroradiometer.

### Test Protocol:

Day 1.

The individual reported to the testing facility and a trained technician determined the initial Minimum Peripheral Pigment Darkening Dose (MPPDu) on a predefined unprotected skin area as follows. A series of 5 UV radiation doses, expressed as Joules/square meter using the recommended geometric progression of 25% incremens, was administered to an unprotected location on the subject's back, between the belt-line and shoulder blades. The 5 dose series, with X representing the amount of UV energy projected to produce the test subject's MPPDu were as shown in Table 5, of Example 4.

Day 2.

The subject returned to the testing facility within 3 to 4 hours following the completion of the MPPD doses for evaluation of the response and to determine the subject's unprotected MPPD (MPPDu) value. The MPPDu value was the smallest UV dose required to produce the first perceptible pigment darkening reaching the sub-site borders, at 2 to 4 hours post-exposure using the following grading scale:

Pigment Grading Scale

| | |
|---|---|
| − | No perceptible pigment darkening |
| ? | Barely perceptible pigment darkening response |
| + | Perceptible persistent pigment darkening reading |
| | the sub-site borders (MPPD) |
| ++ | Moderater pigment darkening with sharp borders |
| +++ | Considerable persistent pigment darkening with |
| | sharp borders to evaluate the effects. |

Two test areas (7.1 cm×7.1 cm), as 50 square centimeter rectangles, were drawn on the designated location of the subject's predefined area of the back using a template and an indelible marker. The trained technician applied a composition of Example 1-B to one of the test areas and a JCIA standard sunscreen in the adjacent test area. Each composition, in an amount of 2 mg/cm², was applied by “spotting” the product across the test area and gently spreading, using a finger cot, until a uniform film was applied to the entire test area. The applied product was allowed to dry a minimum of 15 minutes prior to UV exposure.

The technician administered a series of 5 UV radiation doses, expressed as Joules/square meter using the recommended geometric progression of 25%, where the middle exposure is placed to yield the expected UVA-PF of 8.0 The exact series of exposures was determined by the previously established MPPDu from Day 1 and the expected UVA-PF of the test product. The MPPDp was administered using the dose levels shown in Table 5 of Example 4.

On Day 2, the technician administered a second timed series of 5 UV doses, increasing in 25% increments to an unprotected area of the subject's back to determine the subject's second day MPPDu, the doses including the original MPPDu from Day 1 as shown in Table 5, of Example 4.

Day 3.

The subject returned to the testing facility after 3 to 4 hours following completion of the UV doses from Day 2. The MPPD values for all sites that received UV doses, both protected and unprotected areas, were evaluated and recorded under a source of illumination that was a warm white fluorescent light bulb that provided a level of illumination within the range of 450 to 550 lux. The subject was prone when the test site was irradiated and when evaluated.

The study was conducted in a double-blinded manner. Neither the test subject nor the designated technician who evaluated the MPPD responses knew which formulation was applied to which site or what doses of UV radiation were administered, because different technicians performed the product application and administered the doses of UV radiation.

The UVA-PF values were calculated for both the composition of Example 1-B and the P2 standard for each individual subject by calculating the ratio of the MPPDp (value produced in the sunscreen protected site) to the MPPDu (value produced in the unprotected test area), using the calculation: MPPDp/MPPDu=UVA-PF value for each subject and the mean calculated. The sunscreen drug product was classified into a Category Description for Labeling Purposes as follows: PFA Value 2 or more but less than 4=PA+; 4 or more but less than 8=PA++; and 8 or more=PA+++.

### Results.

The composition of Example 1-B tested under Static Mean UVA-PF value of 10.1 and a Category Description of PA+++, whereas the JCIA certified reference sunscreen had a Static Mean UVA-PF value of 4.2 The data from the ten subjects met the statistical criteria for validity. No adverse experiences were reported during this in vivo study.

### Example 6

### Synergistic Efficacy

This example illustrates the synergistic efficacy of the combination of the complexion modifying components (a) 4-hexylresorcinol, (b) niacinamide and (c) 1-amino-ethylphosphinic acid, compared to the effect of the individual components, based on an in vitro evaluation of melanin production/secretion in melanocytes. Components (a), (b) and (c) were evaluated individually and in combination with one another as described below in Studies I and II, and in Tables 9 and 10.

Study I.

One set of aqueous stock solutions was prepared containing each component individually (as supplied) in an amount representing the active concentration present in a composition of Example 1-B (Compositions 6A-C in Table 9). A corresponding second and third set of stock solutions was prepared, respectively containing two-times (Compositions 6E-G in Table 9) and four-times (Compositions I-K in Table 9) the amount of the foregoing concentrations. Combinations of each component were prepared from the stock solutions of each component (Compositions. 6D, 6H, and 6L in Table 9) to provide amounts representing the weight ratio of each component to one another present (active basis) in a composition of Example 1-B as shown below in Notes 1-3 of Table 9. A 20-fold (20×) dilution (distilled water) of each stock solution was selected for the in vitro test to minimize or avoid cytotoxicity of melanocytes in the cell growth medium.

TABLE 9

| | | % Active Conc. in | |
|---|---|---|---|
| Comp. | Component Material | Stock Soln | 20X Dil. |
| 6A | (a) 4-hexylresorcinol | 0.5 | 0.025 |
| 6B | (b) niacinamide | 1 | 0.05 |
| 6C | (c) 1-amino-ethylphosphinic | 0.225 | 0.01 |
| | acid | | |
| 6D | (a)/(b)/(c) blend (Note 1) | 0.5/1/0.225 | 0.025/0.05/0.01 |
| 6E | (a) 4-hexylresorcinol | 1 | 0.05 |
| 6F | (b) niacinamide | 2 | 0.1 |
| 6G | (c) 1-amino-ethylphosphinic | 0.45 | 0.0225 |
| | acid | | |
| 6H | (a)/(b)/(c) blend (Note 2) | 1/2/0.45 | 0.05/0.1/0.0225 |
| 6I | (a) 4-hexylresorcinol | 2 | 0.1 |
| 6J | (b) niacinamide | 4 | 0.2 |
| 6K | (c) 1-amino-ethylphosphinic | 0.9 | 0.045 |
| | acid | | |
| 6L | (a)/(b)/(c) blend (Note 3) | 2/4/0.9 | 1/0.2/0.045 |

Notes to Table 9:
1. Composition 6D was prepared by combining an amount of the stock solution of Compositions 6A, 6B and 6C having the % active concentration of components (a), (b) and (c) indicated to provide a blend having a ratio of (a)/(b)/(c) of 1/2/0.5 (active weight basis).
2. Composition 6H was prepared by combining an amount of the stock solution of Compositions 6E, 6F and 6G having the % active concentration of components (a), (b) and (c) indicated to provide a blend having a ratio of (a)/(b)/(c) of 1/2/0.5 (active weight basis).
3. Composition 6L was prepared by combining an amount of the stock solution of Compositions 6I, 6J and 6K having the % active concentration of components (a), (b) and (c) indicated to provide a blend having a ratio of (a)/(b)/(c) of 1/2/0.5 (active weight basis).

Component (c) as supplied is described in Note (a) to Table 2. As a positive control, stock solutions of kojic acid solutions were prepared at an active concentration of 2%, 0.5% and 0.1% to provide a concentration of 0.1%, 0.025% and 0.005%, respectively, at a 20× dilution. Distilled water was used as the negative control.

Methodology.

Cells (B16 melanocytes) were plated in phenol-free Dulbecco's Modified Eagle's Medium (DMEM) supplemented with 5% fetal bovine serum (FBS) at 10,000 cells/well. After 24 hours, test material was added and the cells were incubated with the test material for 48 hours. The melanin was quantified by instrumentally measuring absorbance with a BIORAD microplate spectrophotometer 3550-UV at 490 nanometers. Cultures were also photographed (CANON REBEL digital camera). Statistical significance of the values was calculated using paired t-test and threshold statistical significance was fixed at p=0.05 and 15% difference as compare to the water control group. Cell numbers were measured using a sulforhodamine B method (described by Skehan et al, “New colorimetric cytotoxicity assay for anticancer-drug screening”, J. Natl Cancer Inst., 82:1107, 1990, the relevant disclosures of which are incorporated herein by reference). The colorimetric signal proportional to cell numbers was acquired with the microplate spectrophotometer 3550-UV at 550 nanometers.

### Results of Study I:

Component (a) (4-hexylresorcinol) by itself was cytotoxic or cytostatic at all concentrations tested so specific inhibition of melanin production/secretion could not be established. The individual components, (b) (niacinamide) and (c) (1-amino-ethylphosphinic acid), had no effect either on pigmentation or cell numbers at all of the concentrations tested. Surprisingly, the combination of components (a), (b) and (c) (Comp. 6D, Table 9) had a statistically significant effect on the inhibition of melanin production/secretion at the 20× dilution tested (at the other concentrations, the specific inhibition of melanin could not be tested owing to cytotoxicity).

The pigmentation expressed as % was 100% (no inhibition of melanin production) for the water control, and was reduced to 69% by the combination of components (a),(b),(c), (Comp. at 20× dilution), thereby representing about 31% inhibition of melanin production. Additionally, no cytotoxicity was noted. The positive control, kojic acid, exhibited a dose-dependent pigmentation-inhibitory effect, (% pigmentation ranged from 70-90%, as the concentration increased, representing about 10 to about 30% inhibition of melanin production) without significantly impacting cell proliferation thereby technically validating the test.

Study II.

Following the general procedure of Study I, a further set of dilute aqueous stock solutions was prepared containing each component individually (Compositions 6M-O in Table 10). A stock solution containing a combinations of each component was prepared from the stock solutions of each component (Composition 6P in Table 10) to provide amounts representing the weight ratio of each component to one another present in a composition of Example 1-B as shown below in Note 1 to Table 10. For comparison, a dilute stock solution of hydroquinone (Composition 6Q) was included. A 20-fold (20×) dilution (distilled water) of each stock solution was selected for the in vitro test to minimize or avoid cytotoxicity of melanocytes in the cell growth medium.

TABLE 10

| | | % Active Conc. in | |
|---|---|---|---|
| Comp. | Component Material | Stock Soln | 20X Dil. |
| 6M | (a) 4-hexylresorcinol | 0.02 | 0.001 |
| 6N | (b) niacinamide | 0.04 | 0.002 |
| 6O | (c) 1-amino-ethylphosphinic | 0.009 | 0.00045 |
| | acid | | |
| 6P | (a)/(b)/(c) (blend) | 0.02/0.04/ | 0.001/0.002/ |
| | | 0.009 | 0.00045 |
| 6Q | hydroquinone | 0.02 | 0.001 |

Note to Table 10:
1. Composition 6P was prepared by combining an amount of the stock solution of Compositions 6M, 6N and 6O having the % active concentration of components (a), (b) and (c) indicated to provide a blend having a ratio of (a)/(b)/(c) of 1/2/0.5 (active weight basis).

Distilled water was used as the negative control. As a positive control for validating the test, a dilute stock solutions of kojic acid (0.2%, 0.04%) were prepared and a 20× dilution (0.01%, 0.002%) tested.

The methodology of Study I was repeated, except that the test was terminated after 72 hours for quantifying melanin pigmentation.

### Results of Study II:

At the 20× dilution concentration, the individual component (a) (4-hexylresorcinol) was cytotoxic, and the individual components, (b) (niacinamide) and (c) (1-amino-ethylphosphinic acid), had no inhibitory effect on either pigmentation or on cell numbers. Surprisingly, the combination of components (a), (b) and (c) (Comp. 6P, Table 10) had a statistically significant inhibitory effect (about 62%) on melanin production/secretion without any cytotoxic effect. Hence, the combination (Comp. 6P) demonstrated a specific whitening effect which resulted in better whitening and a non-cytotoxic profile than that of the individual components which again was judged synergistic.

Under the test conditions, hydroquinone had a whitening effect of at last about 50% inhibition of melanin secretion, but this benefit was partially offset by browning of the medium due to auto-oxidation. The test was again technically validated by the positive control, kojic acid, which exhibited a strong pigmentation-inhibitory effect (about 73%) without significantly impacting cell proliferation.

In conclusion, the combination of components (a), (b) and (c) (Comp. 6D, Table 9 and Comp. 6P, Table 10) exhibited a surprisingly enhanced inhibitory effect on melanin production (i.e., whitening) and a non-cytotoxic profile than that of the individual components. Hence, the combination of complexion modifying components (a), (b) and (c) representing the ratio of the amounts to one another as present in a preferred composition of this invention was judged synergistic compared to the effect of the individual components.

Preferred embodiments of this invention are described herein, including the best mode known to the inventors for carrying out the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A topical skin care composition comprising, in an aqueous cosmetically acceptable vehicle, the following complexion modifying components (a)-(c):
(a) at least one 4-alkylresorcinol, wherein the alkyl group comprising 1 to 8 carbon atoms;
(b) at least one vitamin selected from the group consisting of vitamin classes A, B, C, and E; and
(c) at least one unsubstituted or substituted alpha-aminoalkylphosphinic compound having the following structural Formula (I): 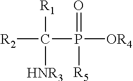
wherein R₁ is a hydrogen atom, a linear alkyl group, a branched alkyl group, or a thiazoline group; R₂ is a hydrogen atom, a linear alkyl group, a branched alkyl group, or R₂ is a linear or branched alkyl group including a substituent selected from a carboxyl, a hydroxyl, an amine, and a thiol group; R₃ is a hydrogen atom, a linear alkyl group, a branched alkyl group, an arylalkyl group, an acyl group, or an acyloxy group; R₄ is a hydrogen atom, a linear alkyl group, or a branched alkyl group; and R₅ is a hydrogen atom, a hydroxyl group, a linear alkyl group, or a branched alkyl group.

2. The composition of claim 1 wherein the alkyl group of component (a) comprises 4 to 6 carbon atoms.

3. The composition of claim 1 wherein component (a) comprises 4-hexylresorcinol.

4. The composition of claim 1 wherein component (b) comprises a B vitamin.

5. The composition of claim 4 wherein component (b) comprises vitamin B₃ (niacinamide).

6. The composition of claim 1 wherein component (c) comprises 1-aminoethylphosphinic acid, 1-amino-3-methylbutylphosphinic acid, or a combination thereof.

7. The composition of claim 6 wherein component (c) comprises 1-aminoethylphosphinic acid.

8. The composition of claim 1 wherein the cosmetic vehicle includes a skin conditioning effective amount of at least one conditioning agent selected from the group consisting of a humectant, an emollient, and a moisturizer.

9. The composition of claim 1 wherein the cosmetic vehicle includes at least one nonionic emulsifier selected from the group consisting of a C₆-C₂₂ fatty acid ester of polyoxyethylene, a monoglyceride, a diglyceride, a sorbitan ester, an ethoxylated sorbitan ester, a C₈-C₂₂ fatty alcohol, an alkoxylated fatty alcohol, and a combination of two or more thereof.

10. The composition of claim 1 wherein the cosmetic vehicle further includes at least one ultraviolet radiation absorbing material.

11. The composition of claim 10 wherein the radiation absorbing material is a UVA absorber.

12. The composition of claim 10 wherein the radiation absorbing material is a UVB absorber.

13. The composition of claim 10 wherein the radiation absorbing material is a broad spectrum absorber.

14. The composition of claim 10 wherein the ultraviolet radiation absorbing material provides a calculated sun protection factor (SPF) value of at least 15.

15. The composition claim 10 wherein the ultraviolet radiation absorbing material provides a calculated sun protection factor (SPF) value of at least 30.

16. The composition of claim 1 wherein the composition has a physiologically tolerable pH in the range of about pH 4 to about pH 8.

17. A topical skin care composition comprising, in an aqueous cosmetically acceptable vehicle:
(a) a combination of complexion modifying components consisting essentially of 4-hexylresorcinol, vitamin B₃ (niacinamide), and 1-amino-ethylphosphinic acid;
(b) at least one skin conditioning agent selected from the group consisting of a humectant, an emollients, and a moisturizer;
(c) at least one skin protective agent selected from the group consisting of UVA, UVB, and broad spectrum ultraviolet radiation absorbing material; and
(d) at least one nonionic emulsifying agent selected from the group consisting of a C₆-C₂₂ fatty acid ester of polyoxyethylene, a monoglyceride, a diglyceride, a sorbitan ester, an ethoxylated sorbitan ester, a C₈-C₂₂ fatty alcohol, an alkoxylated fatty alcohol, and a combination of two or more thereof.

18-19. (canceled)

20. A method of modifying the skin complexion of a subject comprising the step of topically applying the skin care composition of claim 1 at least once to the skin of the subject, spreading the applied composition on the skin, and allowing the applied composition to dry.

21. The skin care composition of claim 17 wherein the composition has a physiologically tolerable pH in the range of about pH 4 to about pH 8.

22. A method of modifying the skin complexion of a subject comprising the step of topically applying the skin care composition of claim 17 at least once to the skin of the subject, spreading the applied composition on the skin, and allowing the applied composition to dry.
